(19) United States
(12) Patent Application Publication
(10) Pub. No.: US 2016/0262636 A1
(43) Pub. Date: Sep. 15, 2016

(54) HEALTH-ABNORMAL CONDITION ALARM SYSTEM USING MULTI BIO-SIGNAL

(71) Applicant: SoSo Co.,Ltd, Gyeongdae-ro (KR)

(72) Inventors: Dong-Bin MIN, Gimpo-si (KR); Jae-Yong LEE, Seoul (KR); Dae-Sik KEUM, Seoul (KR); Yoon-Ha HWANG, Daegu (KR); Won-Pyo KIM, Daegu (KR); Sang-Tak KIM, Daegu (KR); Yoo-Jin JUNG, Daegu (KR)

(73) Assignee: SoSo Co.,Ltd, Gyeongdae-ro (KR)

(21) Appl. No.: 14/842,015

(22) Filed: Sep. 1, 2015

(30) Foreign Application Priority Data
Mar. 13, 2015 (KR) 10-2015-0035158

(51) Int. Cl. A61B 5/0205; A61B 5/00

(52) U.S. Cl. CPC A61B 5/02055; A61B 5/6814; A61B 5/746; A61B 5/0006; A61B 5/0008; A61B 5/02438

(57) ABSTRACT

The present invention relates to a health-abnormal condition alarm system using a multi bio-signal comprising: a wearable device which is configured to be worn on a user's body and senses the multi bio-signal; and a mobile communication terminal receiving a brainwave signal, a heartbeat signal, and a body temperature signal transmitted from the wearable device through a first communications network. The wearable device comprises a headset body, a plurality of electrodes, a brainwave detection unit, a heartbeat detection module, a body temperature detection unit, a headset wireless communication unit, and a headset controller. A health check application is installed in the mobile communication terminal, checks whether the user's health is abnormal or not based on the brainwave signal, the heartbeat signal, and the body temperature signal, and transmits a health-abnormal alarm to a previously-registered external device through a second communications network when the user's health condition is determined as abnormal. Accordingly, a health-abnormal condition alarm system using a multi bio-signal is provided which detects a brainwave, a heartbeat, and a body temperature of a user by using one wearable device, and which transmits an alarm to the outside when a health-abnormal condition of an observed person such as the old and the infirm is appeared by using the detected information, such that can more efficiently manage an emergency or a presymptom of the old and the infirm, etc.

### TECHNICAL FIELD

The invention relates to a health-abnormal condition alarm system using a multi bio-signal and in particular to health-abnormal condition alarm system using a multi bio-signal which can transmits an alarm to the outside by using information such as a brainwave, a heartbeat, and a body temperature when the health-abnormal condition of an observed person such as the old and the infirm is appeared.

### BACKGROUND ART

Recently, a field of a home health care is becoming an issue, in particular, in case of the old and the infirm, such as senior citizen who lives alone, a technical field for checking a health-abnormal at home is widely researched. In one example, in case of a diabetic patient, a technology which can check a blood sugar at home, periodically, and can transmit the checked blood sugar to a hospital, etc. in real time through the portable device which can check the blood sugar is being suggested. “Smart self blood sugar tester and method for measuring blood sugar using mobile communication terminal” is disclosed in Korean laid-open publication No. 10-2014-141104.

The world's major industrialized nations raise a health management problem of the senior citizens as the major issue together with the field of the home health care at the time of entering the aging society. In case of the house where the old man lives alone, nobody deals with the emergency so that the golden time is lost.

In case of the emergency which is occurred to the old man, the presymptom appears mostly. If such presymptom is check in advance, the emergency can be predicted in advance and the protector of the old man can visit the house where the old man lives.

However, the presymptom can be predicted accurately not by only one bio-signal but the multi bio-signal. For that reason, it is inconvenient that various devices for detecting each bio-signal are equipped in home and the old man and the infirm check using the various devices, periodically.

Therefore, if only one device can measure the multi bio-signal and can transmit a measured signal or an abnormal alarm to the hospital or the protector, etc., the emergency can be reduced, remarkably.

### DISCLOSURE OF THE INVENTION

### Technical Problem

An object of the present invention is to solve the above problems and the present invention provides a health-abnormal condition alarm system using a multi bio-signal which can transmits an alarm to the outside by using information such as a brainwave, a heartbeat, and a body temperature when the health-abnormal condition of an observed person such as the old and the infirm is appeared.

### Technical Solution

The above purpose can be achieved by a health-abnormal condition alarm system using a multi bio-signal comprising: a wearable device which is configured to be worn on a user's body and senses the multi bio-signal; and a mobile communication terminal receiving a brainwave signal, a heartbeat signal, and a body temperature signal transmitted from the wearable device through a first communications network; wherein the wearable device comprises: a headset body configured to be mounted on the user's head, a plurality of electrodes which are exposed on an outer surface of the head set body so as to be in contact with a forehead of the user when the headset body is mounted on the user's head, a brainwave detection unit which is embedded in the headset body and which detects the brainwave signal based on a signal detected by the plurality of electrodes, a heartbeat detection module which extends from the headset body through a signal cable and which is configured to be mounted on the user's ear to detect the heartbeat signal, a body temperature detection unit which is installed at the headset body and which detects a body temperature and generates the body temperature signal, a headset wireless communication unit which is embedded in the headset body and which carries out a wireless communication through the first communications network, and a headset controller transmitting the brainwave signal, the heartbeat signal, and the body temperature signal to the mobile communication terminal through the headset wireless communication unit; wherein a health check application is installed in the mobile communication terminal; and wherein the health check application checks whether the user's health is abnormal or not based on the brainwave signal, the heartbeat signal, and the body temperature signal transmitted from the wearable device, and transmits a health-abnormal alarm to a previously-registered external device through a second communications network when the user's health condition is determined as abnormal.

Here, the health check application transmits the health-abnormal alarm the external device when a brainwave based on the brainwave signal deviates from a predetermined reference brainwave over 20%, when a heartbeat based on the heartbeat signal deviates from a predetermined reference heartbeat over 20%, or when the body temperature based on the body temperature signal deviates from a predetermined reference body temperature over 2° C.

Also, the heartbeat detection module is configured in the form of clamps to be mounted on the user's ear.

Also, the health-abnormal condition alarm system using the multi bio-signal further comprises a permanent magnet which is provided on one of an outer surface of the heartbeat detection module and an outer surface of the headset body; and a magnetic substance which is provided on the other of the outer surface of the heartbeat detection module and the outer surface of the headset body and which has magnetic material or magnetizable material. The heartbeat detection module are configured to be attached to and detached from the headset body by the attachment of the permanent magnet and the magnetic substance.

Further, the mobile communication terminal further comprises a health check database; and the health check application stores the brainwave signal, the heartbeat signal and the body temperature signal on the health check database by a predetermined time interval and provides with a health condition information by a predetermined cycle based on data stored in the health check database.

Also, the health check application of the mobile communication terminal provides the user with a diagnosis recommendation alarm, when it is determined as the health-abnormal condition.

### ADVANTAGEOUS EFFECT

According to the present invention, a health-abnormal condition alarm system using a multi bio-signal is provided which detects a brainwave, a heartbeat, and a body temperature of a user by using one wearable device, and which transmits an alarm to the outside when a health-abnormal condition of an observed person such as the old and the infirm is appeared by using the detected information, such that can more efficiently manage an emergency or a presymptom of the old and the infirm, etc.

### BRIEF DESCRIPTION OF THE DRAWINGS

FIG. 1 represents an arrangement of a health-abnormal condition alarm system using a multi bio-signal according to the present invention.

FIGS. 2 to 5 represent an arrangement of a wearable device according to the present invention.

FIG. 6 represents an arrangement of a mobile communication terminal according to the present invention.

### MODE FOR CARRYING OUT THE INVENTION

Hereinafter, embodiments of the present invention will be explained in detail referring to attached drawings.

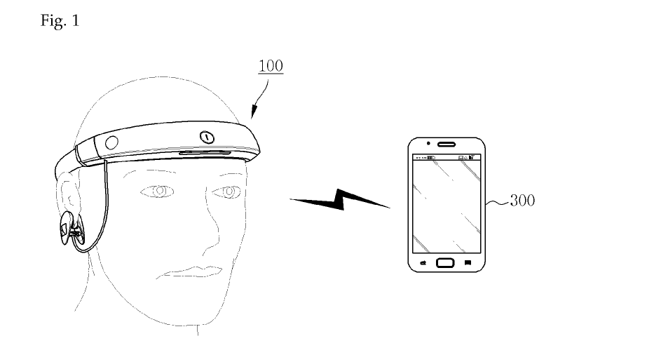

FIG. 1 represents an arrangement of a health-abnormal condition alarm system using a multi bio-signal according to the present invention. Referring to FIG. 1, a health-abnormal condition alarm system comprises a wearable device 100 and a mobile communication terminal 300.

The wearable device 100 is configured to be worn on a user's body, and detects the multi bio-signal when the user wears the wearable device 100. The wearable device 100 is provided as a type of one device for detecting the multi bio-signal. In an embodiment of the present invention, the wearable device 100 may configured to be mounted on a user's head, as shown in FIG. 1.

The mobile communication terminal 300 is connected to the wearable device 100 through a first communication network, and receives the multi bio-signal from the wearable device 100. In an embodiment of the present invention, the wearable device 100 may receive a brainwave signal, a heartbeat signal and a body temperature signal as the multi bio-signal.

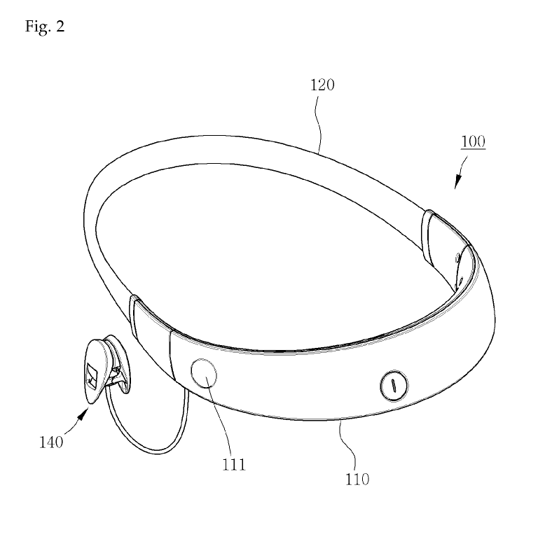

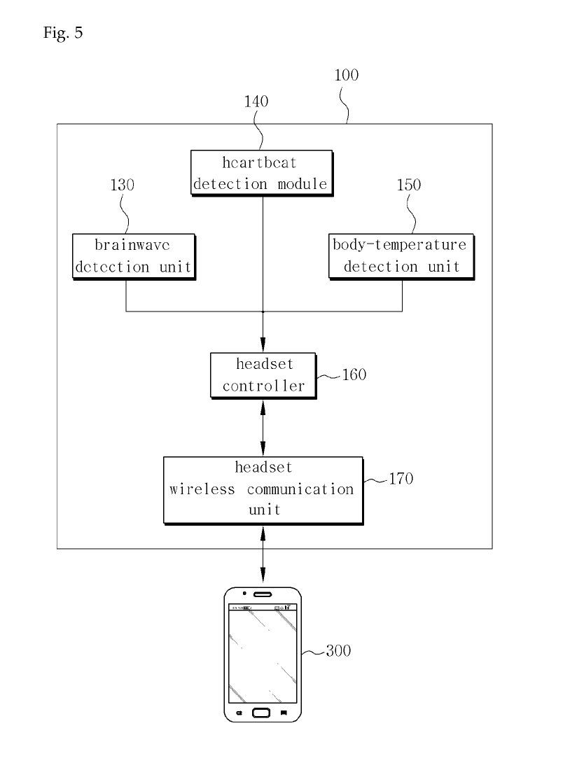

Hereinafter, referring to FIGS. 2 to 5, the arrangement of the wearable device 100 according to the present invention will be explained in detail.

As shown in FIGS. 2 to 5, the wearable device 100 according to the present invention comprises a headset body 110, 120, a plurality of electrodes 131, a brainwave detection unit 130, a heartbeat detection module 140, a body temperature detection unit 150, a headset wireless communication unit 170, and a headset controller 160.

The headset body 110, 120 is configured to be mounted on the user's head. In one example as shown in FIGS. 1 to 4, the headset body 110, 120 is arranged in the form of a headband, but the shape of the headset body is not limited to this.

Further, the headset body 110, 120 comprises a body module 110 in which a circuit arrangement such as the brainwave detection unit 130, the heartbeat detection unit 140, the body temperature detection unit 150, the headset wireless communication unit 170, and the headset controller 160 is embedded and an elastic headset band 120 which is connected to the body module 110. With this arrangement, the elasticity of the headset band 120 allows the headset body 110, 120 to be mounted on the user's head stably regardless of the size of the user's head.

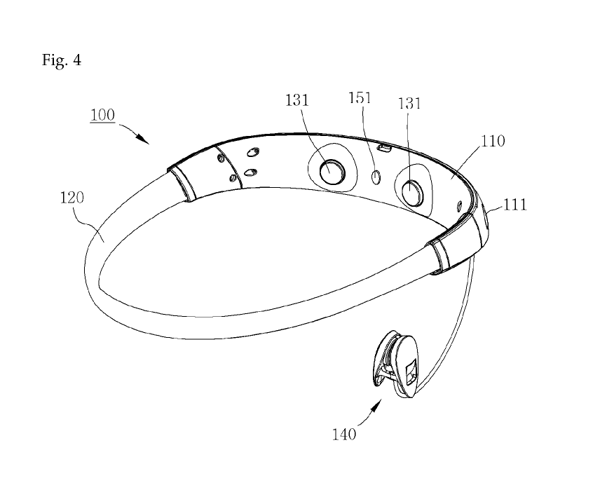

The plurality of electrodes 131 are exposed on the surface of the body module 110 of the headset body 110, 120 such that they are in contact with a user's forehead when the headset body 110, 120 is mounted on the user's head. In an embodiment of the present invention, as shown in FIG. 4, a pair of electrodes 131 is provided, but the number of the electrode is not limited to this.

The brainwave detection unit 130 is embedded in the body module 110 of the headset body 110, 120 such that the brainwave can be determined based on a signal detected by a plurality of electrodes 131.

The heartbeat detection module 140 extends from the headset body 110, 120 by a signal cable and as shown in FIG. 1, it is configured to be mounted on an earlobe so as to detect a user's heartbeat signal.

Herein, the heartbeat detection module 140 is configured in the form of clamps to be mounted on the user's earlobe. As shown in FIG. 1, the user wears the wearable device 100 for detecting the multi bio-signal according to the present invention and mounts the heartbeat detection module 140 which extends through a signal cable on the earlobe, such that the brainwave and the heartbeat can be simultaneously detected by using only one device which is configured in the form of headset.

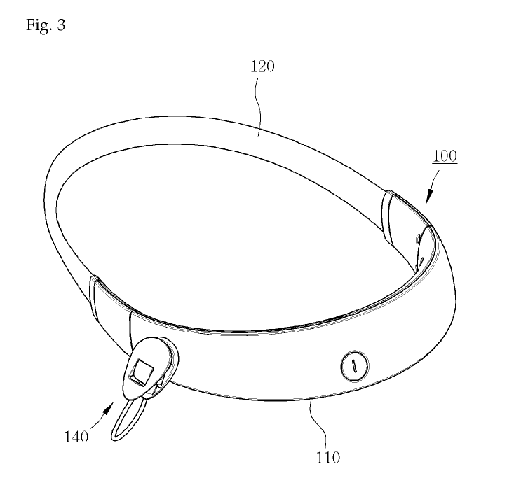

Further, a permanent magnet 111 is mounted on the body module 110 of the headset body 110, 120 and a magnetic substance (not shown) is provided to the heartbeat detection module 140. Here, the magnetic substance can be a magnetic material such as a magnet or a magnetizable material such as a metal. Therefore, as shown in FIG. 3, when a user keeps and stores the wearable device 100 for detecting a multi bio-signal according to the invention, he attaches the magnetic substance of the heartbeat detection module 140 to the permanent magnet 111 of the headset substance 110, 120 in order to keep the apparatus. Here, it is understood that the permanent magnet 111 can be mounted on the heartbeat detection module 140 and the magnetic substance can be provided to the body module 110 of the headset body 110, 120.

The body temperature detection unit 150 detects a user's body temperature through a body temperature detection terminal 151, and generates the body temperature signal. In one embodiment of the present invention, the body temperature detection terminal 151 is formed between the pair of the electrodes 131 such that can detect the body temperature of the user's forehead when the user wears the wearable device 100, as an example.

The headset wireless communication unit 170 is embedded in the body module 110 of the headset body 110,120, and carries out a wireless communication through the first communication network. In one example, the first communication network is provides as a Bluetooth or WIFI network based on TCP/IP. Therefore, the headset wireless communication unit 170 and the mobile communication terminal 100 are connected and communicated to each other through the Bluetooth or WIFI network based on TCP/IP.

The headset controller 160 transmits the brainwave signal detected by the brainwave detection unit 130, the heartbeat signal detected by the heartbeat detection module 140, and the body temperature signal generated by the body temperature detection unit 150 to the mobile communication terminal 300 through the headset wireless communication unit 170.

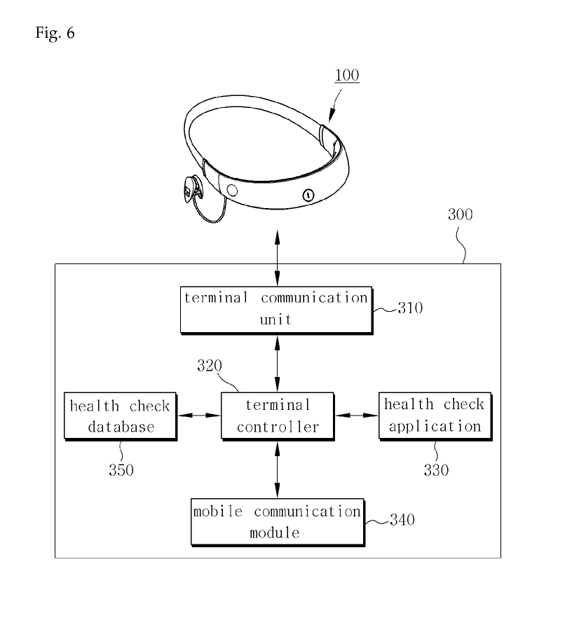

Meanwhile, the mobile communication terminal 300 receives the brainwave signal, the heartbeat signal and body temperature signal from the wearable device 100 through the first communication network. FIG. 6 represents an arrangement of a mobile communication terminal according to the present invention.

Referring to FIG. 6, the mobile communication terminal 300 may comprises a terminal wireless communication unit 310, a mobile communication module 240, a health check application 330, a health check database, and a terminal controller 320.

The terminal wireless communication unit 310 communicates wirelessly with the wearable device 100 through the first communication network. Here, the terminal wireless communication unit 310 receives the brainwave signal, the heartbeat signal and the body temperature single transmitted from the wearable device 100 through the first communication network.

The terminal controller 320 controls the whole function of the mobile communication terminal 100. For example, if the mobile communication terminal 100 is provided as a smart phone, the terminal controller 320 may comprises an application processor (AP), a memory, and an operating system which operates based on the same.

The health check application 330 operates based on the operating system of the mobile communication terminal 100 in the state that installs in the mobile communication terminal 100. Here, the health check application 330 checks whether the user's health is abnormal or not based on the brainwave signal, the heartbeat signal and the body temperature signal which are transmitted from the wearable device 100 and are received through the terminal wireless communication unit 310.

And then, the health check application 330 may transmit a health-abnormal alarm to an external device which is registered previously, when the user's health condition is determined as abnormal. Here, the previously-registered external device may comprise a mobile phone of a protector or a relation, a social worker, a doctor, a server which is managed by a public institution such as a public health center, or a mobile phone of a manager of the public institution, etc.

Here, the mobile communication module 240 communicates with the external device through a second communication network, and the second communication network may comprise a mobile communication network such as 3G or 4G, etc., or WIFI network, etc. Here, the health-abnormal alarm can be transmitted through various methods, such as a text message to the smart phone of the protector or the manager of the public institution, or an alarm through an application of the smart phone, etc.

Meanwhile, the health check application 330 may determine as the health-abnormal condition, when the brainwave of the user based on the brainwave signal deviates from a predetermined reference brainwave over 20%. Also, the health check application 330 may determine as the health-abnormal condition, when the heartbeat of the user based on the heartbeat signal deviates from a predetermined reference heartbeat over 20%.

Also, the health check application 330 may determine as the health-abnormal condition, when the body temperature of the user based on the body temperature signal deviates from a predetermined reference body temperature (36.5° C., for example) over 2° C.

Meanwhile, the health check application 330 may store the brainwave signal, the heartbeat signal and the body temperature signal on the health check database 350 by a predetermined time interval. Therefore, for example, it is possible that a daily or hourly health state of the user is determined easily.

Hereinafter, by the above structure, a method for checking the health-abnormal condition of user using the health-abnormal condition alarm system using the multi bio-signal according to the present invention will be explained. The user such as the old and the infirm prepares the wearable device 100 and the mobile communication terminal 300, and then mounts the wearable device 100 on the head by a predetermined time interval.

For example, the user can mount the wearable device 100 whenever the user gets up every morning, and measure the brainwave, the heartbeat and the body temperature. Generally, when human gets up, the heart beats from the stable state, it is heavy on a cardiovascular system by shifting the posture from a recumbent posture to a standing posture, mental activity is about to begin, and variables for the body temperature will be reduced.

Thus, the multi bio-signal of the use is measured every day, the alarm is transmitted to the protector when the health-abnormal is checked so that the old and the infirm, etc. can be managed more effectively.

The scope of the present invention is not limited to the above described embodiments and is determined by the accompanying claims. It is apparent that various variations or modifications can be made within the scope of the claims by those skilled in the art.

### LIST OF REFERENCE NUMERALS

100: wearable device

110: body module

111: permanent magnet

120: headset band

130: brainwave detection unit

131: electrode

140: heartbeat detection module

150: body temperature detection unit

151: body temperature detection terminal

160: headset controller

170: headset wireless communication unit

300: mobile communication terminal

310: terminal communication unit

320: terminal controller

330: health check application

340: mobile communication module

350: health check database

1. A health-abnormal condition alarm system using a multi bio-signal, comprising:
a wearable device which is configured to be worn on a user's body and senses the multi bio-signal; and
a mobile communication terminal receiving a brainwave signal, a heartbeat signal, and a body temperature signal transmitted from the wearable device through a first communications network;
wherein the wearable device comprises:
a headset body configured to be mounted on the user's head,
a plurality of electrodes which are exposed on an outer surface of the head set body so as to be in contact with a forehead of the user when the headset body is mounted on the user's head,
a brainwave detection unit which is embedded in the headset body and which detects the brainwave signal based on a signal detected by the plurality of electrodes,
a heartbeat detection module which extends from the headset body through a signal cable and which is configured to be mounted on the user's ear to detect the heartbeat signal,
a body temperature detection unit which is installed at the headset body and which detects a body temperature and generates the body temperature signal,
a headset wireless communication unit which is embedded in the headset body and which carries out a wireless communication through the first communications network, and
a headset controller transmitting the brainwave signal, the heartbeat signal, and the body temperature signal to the mobile communication terminal through the headset wireless communication unit;
wherein a health check application is installed in the mobile communication terminal; and
wherein the health check application checks whether the user's health is abnormal or not based on the brainwave signal, the heartbeat signal, and the body temperature signal transmitted from the wearable device, and transmits a health-abnormal alarm to a previously-registered external device through a second communications network when the user's health condition is determined as abnormal.

2. The health-abnormal condition alarm system using the multi bio-signal according to claim 1, wherein the health check application transmits the health-abnormal alarm the external device when a brainwave based on the brainwave signal deviates from a predetermined reference brainwave over 20%, when a heartbeat based on the heartbeat signal deviates from a predetermined reference heartbeat over 20%, or when the body temperature based on the body temperature signal deviates from a predetermined reference body temperature over 2° C.

3. The health-abnormal condition alarm system using the multi bio-signal according to claim 1, wherein the heartbeat detection module is configured in the form of clamps to be mounted on the user's ear.

4. The health-abnormal condition alarm system using the multi bio-signal according to claim 1, further comprising:
a permanent magnet which is provided on one of an outer surface of the heartbeat detection module and an outer surface of the headset body; and
a magnetic substance which is provided on the other of the outer surface of the heartbeat detection module and the outer surface of the headset body and which has magnetic material or magnetizable material,
wherein the heartbeat detection module are configured to be attached to and detached from the headset body by the attachment of the permanent magnet and the magnetic substance.

5. The health-abnormal condition alarm system using the multi bio-signal according to claim 1, wherein the mobile communication terminal further comprises a health check database; and
wherein the health check application stores the brainwave signal, the heartbeat signal and the body temperature signal on the health check database by a predetermined time interval and provides with a health condition information by a predetermined cycle based on data stored in the health check database.

6. The health-abnormal condition alarm system using the multi bio-signal according to claim 1, wherein the health check application of the mobile communication terminal provides the user with a diagnosis recommendation alarm, when it is determined as the health-abnormal condition.
